(19) United States
(12) Patent Application Publication
(10) Pub. No.: US 2023/0295360 A1
(43) Pub. Date: Sep. 21, 2023

(54) METHOD FOR PREPARING POLYION THIN FILM, AND POLYION THIN FILM AND DETECTION KIT

(71) Applicant: Ankon Medical Technologies (Shanghai) Co., LTD., Shanghai (CN)

(72) Inventors: Shupeng ZHANG, Shanghai (CN); Xiaodong Duan, Pleasanton, CA (US)

(73) Assignees: Ankon Medical Technologies (Shanghai) Co., LTD., Shanghai (CN); ANX IP HOLDING PTE. LTD., (omitted)

(21) Appl. No.: 18/017,854

(22) PCT Filed: Jul. 23, 2021
(86) PCT No.: PCT/CN2021/108245

§ 371 Date: Jan. 24, 2023

(30) Foreign Application Priority Data
Jul. 24, 2020 (CN) 202010725943.6

(51) Int. Cl. C08F 220/48; C08J 5/18; C08K 5/46; C08K 5/23; G01N 33/84; G01N 31/22

(52) U.S. Cl. CPC C08F 220/48; C08J 5/18; C08K 5/23; C08K 5/46; G01N 31/221; G01N 33/84; C08J 2333/20

(57) ABSTRACT

The present invention provides a method for preparing a polyion thin film (in particular a polyion thin film with different surface morphologies) and the polyion thin film prepared by the method. The method comprises: providing a substrate, and coating a first lubricant on a surface of the substrate or on a surface of a tinfoil covered on the surface of the substrate; covering a film-forming liquid of the polyion thin film on the surface of the substrate with the first lubricant, and after coating uniformly, covering a cover plate coated with a second lubricant on the film-forming liquid; performing a polymerization reaction of ionic liquid monomers in the film-forming liquid to form a polyion thin film; and separating the polyion thin film and cleaning it to obtain the polyion thin film. The polyion thin films with different surface morphology can be prepared. The present invention further provides a detection kit.

### CROSS-REFERENCE OF RELATED APPLICATIONS

This application claims priority to Chinese Patent Application No. 202010725943.6, filed Jul. 24, 2020, titled “Method for Preparing Polyion Thin Film, Polyion Thin Film and Detection Kit”, all of which are incorporated herein by reference in their entirety.

### FIELD OF INVENTION

The invention relates to the technical field of materials, and particularly relates to a method for preparing a polyion thin film (in particular to a polyion thin film with a patterned surface), a polyion thin film prepared by the method and a detection kit containing the polyion thin film.

### BACKGROUND

Stimulus-sensitive (responsive) materials are materials that respond to changes in an external environment, such as changes in pH, temperature, light, etc., resulting in changes in the microstructure of materials, such as changes in color, and changes in physical and chemical forms. This property makes the stimulus-sensitive materials have great application prospects in sensors, drug release, biological engineering and other aspects. For example, a pH sensitive film, is widely applied in the fields of a pH sensor, drug controlled release, in vivo detection, etc. However, the link between film micromorphology and macroscopic changes (e.g., response rate) has been less studied. How to enlarge the response change and improve the response rate is of great significance for the pH type ionic film to better serve industrial production, but the synthesis of the responsive ionic film with different micromorphology is a difficult problem. Therefore, people are seeking a preparation method which can synthesize pH type ionic films with different surface morphologies, is simple and convenient, and allow repeated use of the films.

In the prior art, in order to prepare an electric film with a pattern, firstly, a polymer mask with a predetermined pattern is formed on a substrate by a dispensing process, then the substrate is subjected to oxygen plasma treatment or ultraviolet irradiation treatment, and a conductive film is prepared on the substrate attached with the polymer mask by a solution process, and finally, the polymer mask is peeled off from the substrate, so that a patterned conductive film is obtained. However, according to this preparation method, only some electric films with simple patterns formed thereon can be obtained, and the pattern accuracy is low. In this method, the electric film is prepared through a dispensing process. Glue itself has ductility or fluidity, which is difficult to control accurately. Due to the influence of dispensing parameters (such as air pressure and dispensing amount), the precision of the formed polymer mask itself is low (of plasma treatment or ultraviolet treatment, the curing rate is affected by the amount of glue, and the precision is higher or lower), which is easy to result in jagged edges, which will affect the precision of the film finally. In addition, the preparation process of the method disclosed in the prior art is difficult to control, the finished product is difficult to peel, and the integrity of the film is difficult to guarantee.

In addition, the preparation of the patterned metal film in the prior art further comprises: firstly, immersing a clean substrate in a dopamine solution until a polydopamine film is formed on the surface of the substrate, taking out the substrate, washing with water and drying with nitrogen to obtain the substrate attached with the polydopamine film; then, placing a photomask on the obtained substrate attached with the polydopamine film, irradiating the substrate for 15 minutes under ultraviolet (UV) light, washing with water and drying with nitrogen to obtain the UV-cured polydopamine substrate; and finally depositing metal ions on the substrate to obtain the patterned metal film. According to this synthesis method, although films with different patterns can be obtained, the steps are too many, a dopamine film needs to be formed first, a photomask is needed, continuous washing with water and nitrogen is needed to ensure the integrity of the pattern of the film, and the patterned film is formed by damaging the polydopamine film through ultraviolet oxidation, resulting in that the process method has low precision and is difficult to produce high-precision patterned films on a large scale.

### SUMMARY OF THE INVENTION

An object of the present application is to provide a simple, convenient and efficient method for preparing a polyion thin film (in particular to a polyion thin film with a patterned surface).

The present application provides a method for preparing a polyion thin film, which comprises the following steps:
step 1, providing a substrate, and coating a first lubricant on a surface of the substrate or on a surface of a tinfoil covered on the surface of the substrate;
step 2, covering a film-forming liquid for the polyion thin film on the surface of the substrate or on the surface of the tinfoil, and after coating uniformly, covering a cover plate coated with a second lubricant on the film-forming liquid;
step 3, performing a polymerization reaction of ionic liquid monomers in the film-forming liquid to form a polyion thin film;
step 4, separating the polyion thin film and cleaning it to obtain the polyion thin film.

Preferably, the substrate comprises a silicon wafer.

Preferably, the silicon wafer is with a patterned surface.

Preferably, the surface of the silicon wafer includes convex ridges, or the surface of the silicon wafer includes concave grooves, or the surface of the silicon wafer is provided with protruding cylinders elements, or the surface of the silicon wafer is provided with circular holes penetrating through the silicon wafer.

Preferably, in the step 1, the first lubricant and the second lubricant are inert to ultraviolet (UV) light and do not interfere with UV light.

Preferably, in the step 1, the first lubricant is selected from one or more of white vaseline, silicone oil, paraffin, mineral oil and grease.

Preferably, in the step 2, the film-forming liquid comprises one or more of an imidazole ionic liquid, a pyridine ionic liquid, a quaternary ammonium salt ionic liquid, a quaternary phosphine ionic liquid and a pyrrolidine ionic liquid.

Preferably, the film-forming liquid comprises one or more of 1-ethyl-3-methylimidazolium tetrafluoroborate, 1-ethyl-3-methylimidazolium chloride, N-ethyl pyridine bromide, tributylmethylammonium chloride, tributylethylphosphonium bromide and N-butyl-N-methyl pyrrolidine bromide.

Preferably, the cover plate is a glass plate or a hard plastic plate that is resistant to ultraviolet light.

Preferably, the second lubricant is selected from the group consisting of white vaseline, silicone oil, paraffin, mineral oil and grease.

Preferably, in the step 3, the polymerization reaction is carried out under irradiation of UV light.

Preferably, the wavelength of the UV light is 250 nm-400 nm.

Preferably, the irradiation time of the UV light is 15 min-30 min.

Preferably, in the step 3, the polymerization reaction is carried out at a temperature of 20° C. -60° C.

Preferably, the polymerization reaction is initiated by an initiator; More preferably, the initiator is selected from photoinitiator 907, photoinitiator 184, azobisisobutyronitrile, 2,4,6-(trimethylbenzoyl) diphenylphosphine oxide or benzoin and derivatives thereof.

Preferably, in the step 4, the step of separating the polyion thin film comprises: placing the cover plate attached with the polyion thin film into water for standing.

Preferably, the cleaning comprises ultrasonic cleaning in clear water, absolute ethyl alcohol and clear water in sequence.

Preferably, the step 1 further comprises: wetting the substrate with a solvent before spreading the tinfoil on the substrate.

Preferably, the solvent is water, ethyl alcohol or a mixed solvent thereof.

Preferably, in the step 2, the film-forming liquid is prepared in the following steps:
step a, fully mixing ionic liquid monomers;
step b, adding a cross-linking agent and an initiator, and then carrying out an ultrasonic treatment to obtain the film-forming liquid.

Preferably, the step a further comprises: performing ultrasonic treatment on obtained mixed solution after the ionic liquid monomers are fully mixed; and more preferably, the ultrasonic treatment is performed for 10 min-30min.

Preferably, in the step b, the ultrasonic treatment is performed for 10 min-30 min.

Preferably, in the step a, the ionic liquid monomers comprises bromobutane, vinylimidazole and acrylonitrile.

Preferably, the cross-linking agent is N, N-methylenebisacrylamide, and the initiator is 2,4,6-(trimethylbenzoyl) diphenylphosphine oxide.

Preferably, a molar ratio of the bromobutane to the vinylimidazole is from 2:1 to 1:1.

Preferably, the mass of acrylonitrile is greater than or equal to the sum of the mass of the bromobutane and the vinylimidazol.

Preferably, in the step b, the mass of the cross-linking agent is 8 wt%-12 wt% of the total mass of the bromobutane, the vinylimidazole and the acrylonitrile, and the mass of the initiator is 0.5 wt%-2 wt% of the total mass of the bromobutane, the vinylimidazole and the acrylonitrile.

Preferably, the film-forming liquid is prepared by the following steps:
mixing the bromobutane and vinylimidazole in equal molar ratio, and carrying out an ultrasonic treatment on obtained mixed solution for 15 min until the two are fully mixed; adding an amount of the acrylonitrile equal to the total mass of the bromobutane and the vinylimidazole;
adding an amount of N, N-Methylenebisacrylamide that is 8 wt% of the total mass of the bromobutane, the vinylimidazole and the acrylonitrile and an amount of 2,4,6-(trimethylbenzoyl) diphenylphosphine oxide that is 1.0 wt% of the total mass of the bromobutane, the vinylimidazole and the acrylonitrile, and then carrying out the ultrasonic treatment for 15 min to obtain the film-forming liquid .

Preferably, the method further comprising: immersing the polyion thin film in a dye solution containing a dye.

Preferably, the dye is selected from one of bromocresol green, cresol red and methyl orange.

Preferably, the dye solution is a solution obtained by dissolving the dye in a mixed solvent of water and ethyl alcohol, wherein in the mixed solvent of water and ethyl alcohol, the volume ratio of water to ethyl alcohol is 4:1-1:8, preferably 1:4.

Preferably, the concentration of the dye in the dye solution is 0.5-8 mg/ml, more preferably 3.95 mg/ml.

A polyion thin film, which is prepared by the method described above.

A detection kit, comprising the polyion thin film.

Preferably, the detection kit further comprises a colorimetric card.

By using a lubricant, the method for preparing polyion thin film provided herein reduces the difficulty of separating the polyion thin film from the substrate. In addition, the use of tinfoil is not only beneficial to accelerating the film-forming speed of the polyion thin film, but also avoids the direct contact between the film-forming liquid and the substrate (such as a silicon wafer) before film-forming, ensures the integrity of the film and the uniformity of the thickness of the film, and greatly reduces the time required for film separation. The present application also uses a patterned silicon wafer as a template to prepare the polyion thin film with patterned surface.

The method disclosed herein is easy to operate, by which the polyion thin film is easy to separate, the film thickness is uniform and adjustable, and the technical threshold is low, so that the method has great application potential.

The polyion thin film with patterned surface of the present application can be applied to at least the following methods.

Firstly, in vivo or in vitro detection of pH value of body fluids. For example, attaching the polyion thin film to a gastroscope, or taking gastric juice out of the body, so that the polyion thin film is contacted with the gastric juice, and observing the color change of the film to determine the pH value in the body.

Secondly, pH dynamic monitoring, for example, for detecting pH values of water in a swimming pool.

Thirdly, environmental detection, for example, the discharge of industrial wastewater can cause pollution of rivers. In this case, the polyion thin film can be fixed in the river, and at the same time, a colorimetric card is set beside the polyion thin film to determine the pH value by observing the color change of the polyion thin film.

It should be understood that the above description and the details to be set forth in the following text are only exemplary, which are not intended to limit the present application.

### BRIEF DESCRIPTION OF THE DRAWINGS

FIG. 1 is a schematic diagram of a silicon wafer surface having convex ridges.

FIG. 2 is a schematic diagram of a silicon wafer surface having concave grooves.

FIG. 3 is a schematic diagram of a silicon wafer surface provided with protruding cylinders.

FIG. 4 is a schematic diagram of a silicon wafer surface provided with circular holes penetrating through the silicon wafer.

FIG. 5 is a micrograph of a polyion thin film prepared by the method of the present application, which has a fine stripe pattern.

FIG. 6 is a micrograph of a polyion thin film prepared by the method of the present application, which has a crater-like pattern.

FIG. 7 is a micrograph of a polyion thin film prepared by the method of the present application, which has a complex pattern.

### DETAILED DESCRIPTION

In order to make the object, technical solution and advantages of the present application more clear, the technical solution of the present application will be clearly and completely described below in combination with the embodiments of the present application. Obviously, the described embodiments are a part of the embodiments of the present application, rather than all the embodiments. All other embodiments obtained by those skilled in the art without creative work based on the technical solutions and embodiments provided in the present application belong to the protection scope of the present application. The embodiments in which specific conditions are not indicated are carried out under conventional conditions or conditions suggested by the manufacturer.

It should be noted that the term “and/or” or the “/” used herein only means an association relationship describing associated objects, indicating that there can be three types of relationships. For example, A and/or B can refer to: only A exists, both A and B exist, and only B exists. The singular forms “a”, “said” and “the” used in the embodiments of the present application and the appended claims are also intended to include plural forms, unless the context clearly indicates other meanings.

The endpoints of ranges and any values disclosed herein are not limited to the precise ranges or values, and such ranges or values should be understood to include values approaching such ranges or values. For numerical ranges, between endpoints of individual ranges, between endpoints or individual point values of individual ranges, and between individual point values may be combined with each other to yield one or more new numerical ranges.

All the technical features and preferred features mentioned herein can be combined with each other to form new technical solutions unless otherwise specified. Unless otherwise defined or indicated, technical and scientific terms used herein have the same meaning as is familiar to those skilled in the art.

In order to solve the above technical problem, the embodiments of the present application provide a method for preparing a polyion thin film, which comprises the following steps:
step 1, providing a substrate, and coating a first lubricant on a surface of the substrate or on a surface of a tinfoil covering the surface of the substrate;
step 2, covering a film-forming liquid of the polyion thin film on the surface of the substrate coated with the first lubricant, and after coating uniformly, covering a cover plate coated with a second lubricant on the film-forming liquid;
step 3, performing a polymerization reaction of ionic liquid monomers in the film-forming liquid to form a polyion thin film;
step 4, separating the polyion thin film and cleaning it to obtain the polyion thin film with a patterned surface.

According to the method, the polyion thin film is formed between the two surfaces coated with the lubricant by using the film-forming liquid of the polyion thin film, realizing a rapid preparation of the polyion thin film.

In the embodiments of the present application, the surface of the substrate may be smooth, or may be uneven and patterned. The lubricant should be applied evenly to obtain a higher quality polyion thin film.

According to the preparation method, the polyion thin films with different surface morphologies can be prepared, and the synthesis process is simple and convenient, with low cost and easiness in large-scale production, and diversified choices are provided for the application of the polyion thin films in the fields of sensors, medical detection, etc.

In one embodiment of the present application, the substrate comprises a silicon wafer, that is, a silicon wafer is used as the substrate or a substrate assembly containing the silicon wafer is used. Using a silicon wafer as the substrate has the following advantages: the silicon wafer has the inert property, is not sensitive to environmental factors such as temperature, ultraviolet (UV) lights and the like, and has excellent stability; does not interfere with chemical components such as film-forming liquid and the like, and does not influence the curing and film-forming process; the surface of the silicon wafer is easy to control, and patterning can be carried out on the surface, so that the precision of the polyion thin film is improved mainly because the silicon wafer is used in combination with the method of the present application, and the film-forming liquid is solidified to form a film in a determined morphology. Meanwhile, due to the pressing of the cover plate, the film-forming liquid can be evenly spread, so that the overall thickness of the film (i.e. the polyion thin film) can be controlled, and the phenomenon that the film thickness is too low or too high cannot occur. According to different production requirements, in order to meet the requirements of polyion thin films with different thicknesses, the preparation method of the polyion thin film provided by the present application can select cover plates with different thicknesses or different qualities, so as to adjust the thickness of the film. It should be noted that the thickness of the cover plate should be within the range allowing UV light to penetrate through the cover plate, which is not described here.

In the embodiments of the present application, the silicon wafer may be one with a smooth surface or one with a patterned surface. The response speeds of different patterned films are different during detection, and can be adjusted and controlled as required in practical application. Specifically, the ion exchange rate of the films with different patterns prepared according to the substrate of silicon wafer with the patterns is different, and the ion exchange rate can affect the response rate and uniformity of the color development of the films. In particular, for some fields (such as in vivo detection) where higher or finer sensitivity is required but the application of traditional sensors is limited (e.g., limited by volume), the film prepared by the method of the present application can show great advantages.

The pattern on the surface of the silicon wafer can be varied. For example, the surface of the silicon wafer may have convex ridges, concave grooves, cylinders protruding from the surface of the silicon wafer, or circular holes penetrating through the silicon wafer. The individual components forming the pattern (e.g., ridges, grooves, cylinders, or circular holes) may have different size or pitches. For example, the surface of the silicon wafer is engraved with fine stripes, and the gap and size of the stripes can be adjusted, for example, both the gap and size of the stripes can be adjusted to micron level (for example, 50 µm) or millimeter level.

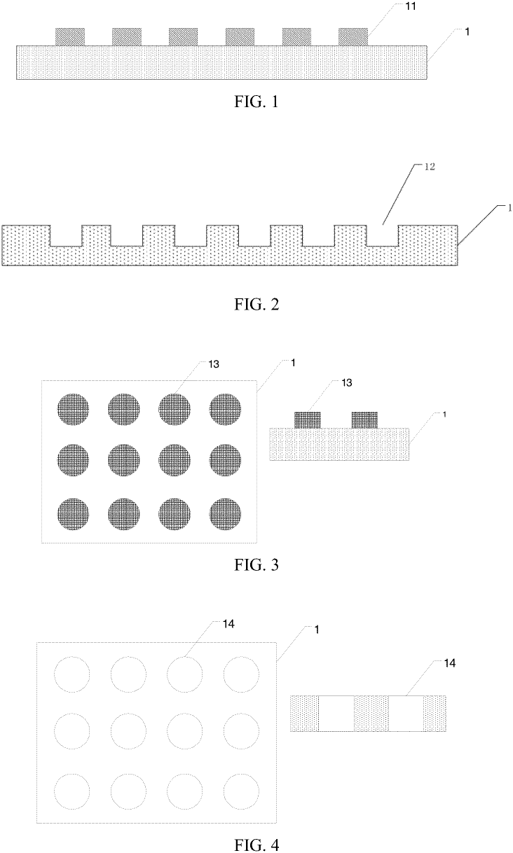

As shown in FIG. 1, the surface of the silicon wafer has convex ridges.

As shown in FIG. 2, the surface of the silicon wafer has concave grooves.

As shown in FIG. 3, the surface of the silicon wafer is provided with protruding cylinders.

As shown in FIG. 4, the surface of the silicon wafer is provided with circular holes penetrating through the silicon wafer.

In one embodiment of the method of the present application, a first lubricant is applied to the surface of the tinfoil. The tinfoil has strong inertia, is not easy to react with a lubricant or a film-forming liquid of the polyion thin film, and has a smooth surface, which is beneficial to forming a more uniform polyion thin film. During the separation of the polyion thin film, the tinfoil can be easily peeled off from the film (even manually torn off), and the side of the resulting polyion thin film in contact with the cover plate can be separated in a liquid environment, thus shortening the separation time.

In a preferred embodiment, the method comprises the steps of:
step 1, laying the tinfoil on a patterned silicon wafer, smoothing the tinfoil, and applying the first lubricant on the surface of the tinfoil far away from the silicon wafer;
step 2, covering the surface of the tinfoil with the film-forming liquid for preparing the polyion thin film, coating uniformly, and covering a cover plate coated with the second lubricant in advance on the tinfoil carrying the film-forming liquid, where the surface of the cover plate coated with the second lubricant is in contact with the film-forming liquid on the tinfoil;
step 3, performing a polymerization reaction of the ionic liquid monomers in the film-forming liquid to form a polyion thin film;
step 4, separating the polyion thin film from the cover plate, and cleaning it to obtain the polyion thin film with a patterned surface.

In the step 1 of an embodiment of the method, the first lubricant is inert to UV light and does not interfere with UV light. More preferably, in the step 1, the first lubricant may be white vaseline, silicone oil, paraffin, mineral oil or grease, etc. The first lubricant needs to be non-toxic, corrosion-free, residue-free and transparent. In the field of medical devices, especially in the fields with high requirements for safety, such as sensors and drug controlled release, the first lubricant has higher requirements, so it is necessary to use medical-grade lubricants. Preferably, in the step 1, the first lubricant is selected from one or more of white vaseline, silicone oil, paraffin, mineral oil and grease. Similarly, the second lubricant is selected from one or more of white vaseline, silicone oil, paraffin, mineral oil, grease.

According to one embodiment of the present application, in the step 2 of the above method, different film-forming liquids may be selected according to different requirements and application scenarios. According to the components of the ionic liquid in the film-forming liquid, the film-forming liquid may comprise one or more of an imidazole ionic liquid, a pyridine ionic liquid, a quaternary ammonium salt ionic liquid, a quaternary phosphine ionic liquid or a pyrrolidine ionic liquid. According to the function performed by the ionic liquid in the film-forming liquid, the film-forming liquid may comprise functionalized ionic liquids commonly used in the art, such as the imidazole ionic liquid (1-ethyl-3-methylimidazolium tetrafluoroborate, 1-ethyl-3-methylimidazolium chloride), the pyridine ionic liquid (N-ethylpyridinium bromide), the quaternary ammonium salt ionic liquid (tributylmethylammonium chloride), the quaternary phosphine ionic liquid or the pyrrolidine ionic liquid (tributylethylphosphonium bromide, N-butyl-N-methylpyrrolidine bromide).

Preferably, in an embodiment, according to the step 2 of the above method, the cover plate may be a transparent substrate. When the substrate is a transparent substrate, the cover plate may be a transparent glass plate or a hard plastic plate that is resistant to UV light (does not react with UV light and UV light can pass through). The cover plate is a glass plate or an UV-resistant transparent hard plastic plate. The transparent glass plate or the UV-resistant transparent hard plastic plate enables the polymerization reaction to be carried out under the irradiation of UV light, and provides an alternative option for the polymerization reaction. In the present application, the UV-resistant transparent hard plastic plate means that the material in the transparent plastic plate is not sensitive to UV light, and the transparent plastic plate cannot be denatured due to the penetration of the UV light. For example, the material for preparing the transparent plastic plate is PC (Polycarbonate), TPU (Thermoplastic polyurethanes), acrylic, and the like.

Preferably, in this embodiment, the second lubricant is inert to UV light and does not interfere with UV light, and is selected from one or more of white vaseline, silicone oil, paraffin, mineral oil and grease.

In the step 3 of the method of an embodiment of the present application, the polymerization reaction is performed under irradiation of UV light. Preferably, the wavelength of the UV light is 250 nm to 400 nm. Preferably, the irradiation time of UV light is 15 min-30 min.

In an embodiment, the polymerization reaction is carried out under heating, and the polymerization reaction is carried out at a temperature of 20° C. -60° C.

In another embodiment, the polymerization reaction is initiated by an initiator in order to facilitate the polymerization reaction. The initiator may be a common initiator in the art, such as photoinitiator 907 (2-methyl-1-(4-methylthiophenyl)-2-morpholine-1-propanone), photoinitiator 184 (1-hydroxycyclohexyl phenyl ketone), azobisisobutyronitrile, 2,4,6-(trimethylbenzoyl) diphenylphosphine oxide (TPO), benzoin and its derivatives, etc. A person skilled in the art may select an appropriate initiator according to the specific components of the film-forming liquid.

Preferably, the polyion thin film is separated from the cover plate by putting the cover plate attached with the polyion thin film into water and standing. In such a way, the polyion thin film can be automatically separated from the cover plate, which is simple in operation while improving the yield rate.

In the step 4 of this embodiment, the cleaning comprises ultrasonic cleaning in clear water, absolute ethyl alcohol and clear water in sequence.

It is worth mentioning that the step 1 of the embodiment of the present application further comprises wetting the substrate with a solvent before laying the tinfoil on the substrate. The purpose of wetting the substrate is to remove the air between the substrate and the tinfoil, and increase the bonding force between the substrate and the tinfoil through the adhesion of the solvent and the tinfoil, so that the tinfoil is easier to smooth and the flatness of the surface of the tinfoil is improved. There may be various solvents for wetting the substrate, but the solvent is preferably water, ethyl alcohol, or a mixed solvent thereof from the viewpoint of source, cost, and environmental protection.

In the step 2 of an embodiment, the film-forming liquid may be prepared by the following steps:
step a, fully mixing the ionic liquid monomers;
step b, adding a cross-linking agent and an initiator, and then carrying out an ultrasonic treatment to obtain the film-forming liquid.

Preferably, in the embodiments of the present application, the step a further comprises the step of performing ultrasonic treatment on obtained mixed solution after the ionic liquid monomers are fully mixed. More preferably, the ultrasonic treatment is performed for 10 min-30 min.

Preferably, in the step b, the ultrasonic treatment is performed for 10 min-30 min.

In the above step a, the ionic liquid monomers comprise bromobutane, vinylimidazole and acrylonitrile. Preferably, the cross-linking agent is N, N-Methylenebisacrylamide (MBA) and the initiator is 2,4,6-(trimethylbenzoyl) diphenylphosphine oxide (TPO).

Specifically, in this embodiment, a molar ratio of bromobutane to vinylimidazole is from 2:1 to 1:1; preferably 1:1. Considering the reaction ratio, conversion rate, dosage and subsequent cleaning process of the two, the molar ratio of bromobutane to vinylimidazole is 1:1.

Preferably, in this embodiment, in the step a, the mass of acrylonitrile is greater than or equal to the sum of the mass of bromobutane and vinylimidazole to ensure the completion of the reaction. Considering the reaction ratio, conversion rate, dosage and subsequent cleaning process of the three components, the mass of the acrylonitrile is the sum of the mass of the bromobutane and vinylimidazole.

Preferably, in the step b of the method provided by the present application, the mass of the cross-linking agent is 8 wt %-12 wt% of the total mass of bromobutane, vinylimidazole and acrylonitrile. For example, it may be 8 wt%, 9 wt%, 10 wt%, 12 wt%, etc. The mass of the initiator is 0.5 wt%-2 wt% of the total mass of bromobutane, vinylimidazole, and acrylonitrile. For example, it may be 0.5 wt%, 1 wt%, 1.5 wt%, 2 wt%, etc.

Specifically, in the step 2 of the method provided by this embodiment, the film-forming liquid may be prepared by the following steps:
step a, mixing the bromobutane and the vinylimidazole in equal molar ratio, and carrying out an ultrasonic treatment on obtained mixed solution for 15 min until the two are fully mixed; adding an amount of acrylonitrile equal to the total mass of the bromobutane and the vinylimidazole; if there are floating impurities on the surface of the mixed solution, the impurities can be removed through a suction tube or a pipette;
step b, adding an amount of MBA that is 8 wt% of the total mass of the bromobutane, the vinylimidazole and the acrylonitrile and an amount of TPO that is 1.0 wt% of the total mass of the bromobutane, the vinylimidazole and the acrylonitrile, and then carrying out an ultrasonic treatment for 15 min to obtain the film-forming liquid.

In the step a, the ultrasonic treatment can increase the energy field for the mixed solution of bromobutane and vinylimidazole, thus accelerating the reaction.

The method provided by the embodiment of the invention further comprises the step of immersing the obtained polyion thin film with the patterned surface in a dye solution with dye, so as to obtain the polyion thin film which can be used for pH detection.

The dye used above may be a common dye in the art as long as it can be stably combined with the film substrate and the safety is guaranteed. For example, the dye may be one selected from bromocresol green (BG), cresol red (CR), methyl orange (MO). After the micromolecule dye BG, CR or MO is exchanged with the film, the micromolecule dye BG, CR or MO has response to the pH value, presents different molecular structures under different pH conditions, microscopically shows different UV spectrum absorption peaks, and macroscopically shows different colors.

Preferably, the dye solution is a solution in which the dye is dissolved in a water-ethyl alcohol mixed solvent, wherein the volume ratio of water to ethyl alcohol in the water-ethyl alcohol mixed solvent is 4:1-1:8. For example, the ratio of water to ethyl alcohol is 4:1, 2:1, 1:1, 1:2, 1:4, 1:6, or 1:8.

Preferably, in the dye solution, the concentration of the dye is 0.5-8 mg/ml. For example, the concentration of the dye is 0.5 mg/ml, 1.0 mg/ml, 1.5 mg/ml, 2.0 mg/ml, 2.5 mg/ml, 3.0 mg/ml, 3.5 mg/ml, 4.0 mg/ml, 4.5 mg/ml, 5.0 mg/ml, 5.5 mg/ml, 6.0 mg/ml, 6.5 mg/ml, 7.0 mg/ml, 7.5 mg/ml or 8.0 mg/ml. Preferably, the concentration of the dye is 3.95 mg/ml.

Therefore, the present application further provides a polyion thin film which is obtained by the preparation method in the above embodiment.

Therefore, the present application further provides a detection kit, which comprises the polyion thin film obtained after the dye solution is impregnated.

In one embodiment of the preferred detection kit of the present application, the detection kit comprises a colorimetric card and the above-mentioned polyion thin film. According to the color change of the polyion thin film, the pH value of the liquid to be detected can be quickly detected by comparing with a pre-made colorimetric card.

The process of preparing the polyion thin film and the specific applications of the polyion thin film are described in detail below with specific examples.

### Embodiment 1

Bromobutane and vinylimidazole with a molar ratio of 2:1 are placed in a glass bottle, and an ultrasonic treatment is performed for 10 min until the two are fully mixed. When oil-like foam impurities floating in a solution can be seen, after removing the impurities by a pipette, acrylonitrile with the same mass as that of bromobutane and vinylimidazole is added, and then MBA (cross-linking agent) with a mass ratio of 8% of the total mass of bromobutane, vinylimidazole and acrylonitrile and TPO (photoinitiator for polymerization reaction) with a mass ratio of 1.0% of the total mass of bromobutane, vinylimidazole and acrylonitrile are added, and then an ultrasonic treatment is performed for 10 min to form a clear and transparent film-forming liquid for polyion thin film.

A transparent glass plate is taken, white vaseline is evenly applied on one surface of the glass plate, the film-forming liquid is drawn and covered on the smooth transparent glass plate, and a transparent glass plate is covered (the surface of the transparent glass plate in contact with the film-forming liquid is coated with silicone oil), and the transparent glass plate is gently pressed. A combination of the two glass plates is irradiated under 250 nm UV light for 15 min (ambient temperature is 20° C.) to initiate the polymerization reaction between bromobutane, vinylimidazole and acrylonitrile to form a transparent film. After the polymerization reaction, the combination is cooled to room temperature. The combination adhered with the transparent film is put into water and immersed for 20 min, and then the polyion thin film is automatically peeled off from between the two transparent glass plates. Subsequently, the polyion thin film is ultrasonically cleaned with pure water, and after the ultrasonic cleaning is finished, the polyion thin film is ultrasonically cleaned with pure water, absolute ethyl alcohol and pure water in sequence, and then blown dry to spare.

### Embodiment 2

Bromobutane and vinylimidazole with the molar ratio of 1:1 are placed in a glass bottle and mixed evenly, then an ultrasonic treatment is performed for 30 min until the two are fully mixed. After removing impurities, acrylonitrile with the same mass as bromobutane and vinylimidazole is added, and then MBA (cross-linking agent) with a mass ratio of 8% of the total mass of bromobutane, vinylimidazole and acrylonitrile and TPO (photoinitiator) with a mass ratio of 1% of the total mass of bromobutane, vinylimidazole and acrylonitrile are added, and then an ultrasonic treatment is performed for 20 min to form a clear and transparent film-forming liquid for polyion thin film.

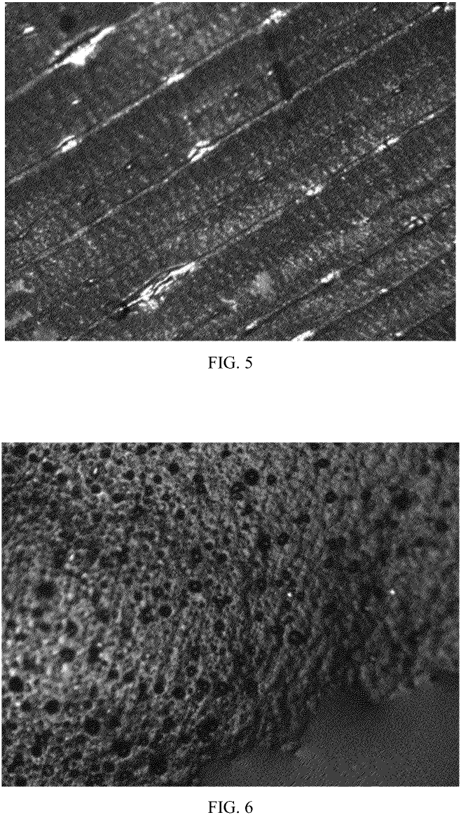

A piece of patterned (such as fine stripes) and cleaned silicon wafer (crystal orientation N <100>) is taken, the silicon wafer is cleaned, the surface of the silicon wafer is wetted using a mixed solvent of water/ethyl alcohol (volume ratio 1:1), the silicon wafer is wrapped with a clean and flat tinfoil, the tinfoil is smoothed out, and then paraffin is evenly coated on the tinfoil. A film-forming liquid is drawn and covered on the silicon wafer, and covered with a transparent glass cover plate (the surface of the glass cover plate contacting with the tinfoil is coated with white vaseline). A formed combination is put under UV light at 265 nm, and is irradiated from the side of the transparent glass cover plate for 20 min (ambient temperature is 55° C.), to initiate the polymerization reaction between bromobutane, vinylimidazole and acrylonitrile to form a transparent film. After the polymerization reaction, the combination is cooled to room temperature and then immersed in water for 20 min, the polyion thin film is automatically peeled off from the glass cover plate. Then, the polyion thin film is ultrasonically cleaned with pure water. After the ultrasonic cleaning is finished, the polyion thin film is immersed in a BG dye solution (the concentration of BG dye is 8 mg/ml, the solvent is a mixed solvent of water and ethyl alcohol, and the volume ratio of water and ethyl alcohol is 1:4), the polyion thin film is placed at 30° C. for 20 min, and the polyion thin film is taken out. At this point, the polyion thin film is blue. Then, the polyion thin film is ultrasonically cleaned with pure water, absolute ethyl alcohol and pure water in turn. It can be seen under microscope (as shown in FIG. 5) that the polyion thin film presents fine stripes, and then is blown dry for later use. The polyion thin film impregnated with BG dye can be used to detect the pH value of a solution.

### Embodiment 3

Bromobutane and vinylimidazole with a molar ratio of 1:1 are placed into a glass bottle, an ultrasonic treatment is performed for 20 min until the two are fully mixed, then acrylonitrile with the same mass as bromobutane and vinylimidazole is added, and then MBA with the mass ratio of 12% of the total mass of bromobutane, vinylimidazole and acrylonitrile and TPO with a mass ratio of 2% of the total mass of bromobutane, vinylimidazole and acrylonitrile are added, and then an ultrasonic treatment is performed for 30 min to form a clear and transparent film-forming liquid for polyion thin film.

A piece of silicon wafer with flat surface (crystal orientation N<100>) is taken, the silicon wafer is cleaned, the surface of the silicon wafer is wetted using with a mixed solvent of water and ethyl alcohol (volume ratio is 1:1), the silicon wafer is wrapped with a clean and flat tinfoil, the tinfoil is smoothed out, and white vaseline is evenly applied on the tinfoil. A film-forming liquid is drawn and covered on the silicon wafer, and covered with a glass cover plate (the surface of the glass cover plate contacting with the tinfoil is coated with white vaseline). A formed combination is put under UV light at 365 nm for 30 minutes (at an ambient temperature of 55° C.) to initiate a polymerization reaction between bromobutane, vinylimidazole, and acrylonitrile to form a transparent film. After the polymerization reaction, the combination is cooled to room temperature and then immersed in water for 25 min, the polyion thin film is automatically peeled off from the glass cover plate. Then, the polyion thin film is ultrasonically cleaned with pure water. After the ultrasonic cleaning is finished, the polyion thin film is immersed in BG dye solution (the concentration of BG dye is 0.5 mg/ml, the solvent is a mixed solvent of water and ethyl alcohol, and the volume ratio of water and ethyl alcohol is 1:1), the polyion thin film is placed at 30° C. for 20 min, and the polyion thin film is taken out. At this point, the polyion thin film is sky blue. Then, the polyion thin film is ultrasonically cleaned with pure water, absolute ethyl alcohol and pure water in turn. It can be seen under microscope that the polyion thin film presents a flat surface, and then is blown dry for later use.

### Embodiment 4

Bromobutane and vinylimidazole with a molar ratio of 1.5:1 are placed into a glass bottle and mixed evenly, then an ultrasonic treatment is performed for 15 min until the two are fully mixed. Then acrylonitrile with the same mass as bromobutane and vinylimidazole is added, and then MBA with a mass ratio of 8% of the total mass of bromobutane, vinylimidazole and acrylonitrile and TPO with a mass ratio of 0.5% of the total mass of bromobutane, vinylimidazole and acrylonitrile are added, and then an ultrasonic treatment is performed for 10 min to form a clear and transparent film-forming liquid for polyion thin film.

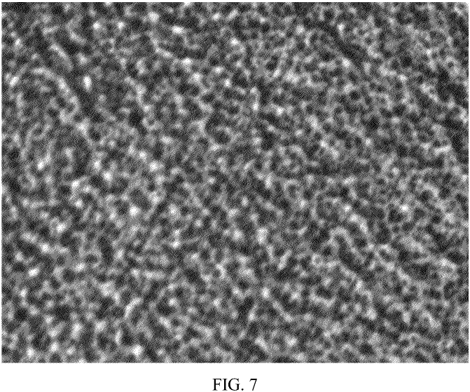

A piece of silicon wafer with a concave surface (crystal orientation N<100>, pattern is self-designed) is taken, the silicon wafer is cleaned, then the surface of the silicon wafer is wetted with a mixed solvent of water and ethyl alcohol (volume ratio is 1:1), the silicon wafer is wrapped with a clean and flat tinfoil, the tinfoil is smoothed out, and then white vaseline is evenly applied on the tinfoil. A film-forming liquid is drawn and covered on the silicon wafer, and covered with a UV-resistant transparent hard plastic plate (the surface of the plastic plate contacting with the tinfoil is coated with white vaseline). A formed combination is put under UV light at 254 nm for 15 min (at an ambient temperature of 55° C.) and irradiated from the side of the transparent hard plastic plate to initiate the polymerization reaction between bromobutane, vinylimidazole, and acrylonitrile to form a transparent film. After the polymerization reaction, the combination is cooled to room temperature and then immersed in water for 20 min, the polyion thin film is automatically peeled off from the glass cover plate. Then, the polyion thin film is ultrasonically cleaned with pure water. After the ultrasonic cleaning is finished, the polyion thin film is immersed in a CR dye solution (the concentration of CR dye is 3.95 mg/ml, the solvent is a mixed solvent of water and ethyl alcohol, and the volume ratio of water and ethyl alcohol is 4:1), the polyion thin film is placed at 30° C. for 20 min, and the polyion thin film is taken out. At this point, film is dark blue. Then, the polyion thin film is ultrasonically cleaned with pure water, absolute ethyl alcohol and pure water in turn. Under the microscope, it can be seen that the polyion thin film presents a concave shape (as shown in FIG. 6), and then is blown dry for later use. The polyion thin film impregnated with CR dye can be used to detect the pH value of a solution.

### Embodiment 5

Bromobutane and vinylimidazole with a molar ratio of 1:1 are placed into a glass bottle and mixed evenly, then an ultrasonic treatment is performed for 15 min until the two are fully mixed. Then acrylonitrile with the same mass as bromobutane and vinylimidazole is added, and then MBA with a mass ratio of 10% of the total mass of bromobutane, vinylimidazole and acrylonitrile and TPO with a mass ratio of 1.0% of the total mass of bromobutane, vinylimidazole and acrylonitrile are added, and then an ultrasonic treatment is performed for 15 min to form a clear and transparent film-forming liquid for polyion thin film.

A piece of silicon wafer (crystal orientation N<100>, pattern is self-designed) engraved with complex pattern is taken, the silicon wafer is cleaned, the silicon wafer is wrapped with a clean and flat tinfoil, the tinfoil is smoothed out, and then mineral oil is evenly applied on the tinfoil. A film-forming liquid is drawn and covered on the silicon wafer, and covered with a glass cover plate (the surface of the glass cover plate contacting with the tinfoil is coated with grease). A formed combination is put under UV light at 254 nm for 15 minutes (at an ambient temperature of 55° C.) to initiate a polymerization reaction between bromobutane, vinylimidazole, and acrylonitrile to form a transparent film. After the polymerization reaction, the combination is cooled to room temperature and then immersed in water for 20 min, the polyion thin film is automatically peeled off from the glass cover plate. Then, the polyion thin film is ultrasonically cleaned with pure water. After the ultrasonic cleaning is finished, the polyion thin film is immersed in a MO dye solution (the concentration of the MO dye is 3.95 mg/ml, the solvent is a mixed solvent of water and ethyl alcohol, and the volume ratio of water and ethyl alcohol is 1:8), the polyion thin film is placed at 30° C. for 20 min, and the polyion thin film is taken out. At this point, the polyion thin film is dark blue. Then, the polyion thin film is ultrasonically cleaned with pure water, absolute ethyl alcohol and pure water in turn. It can be seen under microscope (as shown in FIG. 7) that the polyion thin film presents a pattern corresponding to that of the silicon wafer, and then is blown dry for later use. The polyion thin film impregnated with MO dye can be used to detect the pH value of a solution.

### Application Embodiment 1

This application embodiment describes the application of the polyion thin film prepared by the method of the present application in body fluid detection.

Specifically, a gastric tube is inserted into the stomach of a patient, and 20ml of gastric juice is drawn by a vacuum suction pump. The gastric juice is transferred to a clean beaker, the polyion thin film prepared as in the previous example (impregnated with CR dye) is added and left to stand. Then the polyion thin film is taken out, the color change of the polyion thin film is observed, and the pH value of the gastric juice is obtained by comparing the changed color of the polyion thin film with a colorimetric card. Generally, the pH value of gastric juice is between 2 and 3. If the measured pH value exceeds the reference range of normal value, it can be preliminarily inferred that the patient’s stomach may have lesions.

### Application Embodiment 2

This application embodiment describes the application of the polyion thin film prepared by the method of the present application in water quality detection.

Specifically, the polyion thin film prepared by the method can be used for detecting the pH value of water. pH value is an important index of water quality, which determines many chemical and biological processes in water and is a barometer of water quality. Too high or too low pH value has direct harm to fish and shrimp. Too low pH value can reduce the pH value of fish and shrimp blood, weaken its oxygen-carrying capacity, and may cause physiological hypoxia of fish and shrimp. At this time, although the dissolved oxygen in the water is sufficient, the fish and shrimp may still float due to physiological hypoxia and stop eating. Too high pH value may corrode gill tissue, which may cause mass mortality of fish and shrimp. Too high or too low pH value may inhibit the activities of microorganisms in water and make organic substances difficult to decompose. When the pH value is higher than 8.0, large amounts of ionic ammonia are converted to toxic NH₃; when it is lower than 6.0, more than 90% of sulfides in water can be converted into H₂S, which increases the toxicity of sulfide. Fish and shrimp cannot survive when pH value is lower than 4.0 or higher than 10.6. Therefore, the real-time detection of pH value is very important. In this application embodiment, the polyion thin film (with porous structure) prepared by the application is placed in water, and the pH value of water at this time is determined through the real-time color change of the polyion thin film, and the operation is very simple. Compared with pH test paper, there is no need to take samples for testing and no need to worry about the pollution of water caused by dye spillage; compared with electrochemical sensors, it is cheaper to use the polyion thin film of this application.

The person skilled in the art realizes that the present application by no means is limited to the preferred embodiments described above. On the contrary, many modifications and variations are possible within the scope of the present application. Any modifications, equivalent substitutions, improvements, etc. made within the spirit and principles of this disclosure are intended to be included within the scope of the present invention.

It should be noted that part of the patent application document contains the content protected by copyright. In addition to making copies of the patent documents of the Patent Office or recorded patent documents, the copyright is reserved by the copyright owner.

1. A method for preparing a polyion thin film, comprising:
providing a substrate, and coating a first lubricant on a surface of the substrate or on a surface of a tinfoil covered on the surface of the substrate;
covering a film-forming liquid of the polyion thin film on the surface of the substrate or on the surface of the tinfoil, and after coating uniformly, covering a cover plate coated with a second lubricant on the film-forming liquid;
performing a polymerization reaction of ionic liquid monomers in the film-forming liquid to form the polyion thin film;
separating the polyion thin film and cleaning it to obtain the polyion thin film.

2. The method of claim 1, wherein the substrate comprises a silicon wafer, which is the silicon wafer with a patterned surface;
or, the surface of the silicon wafer comprises convex ridges, or the surface of the silicon wafer comprises concave grooves, or the surface of the silicon wafer is provided with protruding cylinders, or the surface of the silicon wafer is provided with circular holes penetrating through the silicone sheet.

3. The method of claim 1, wherein the first lubricant and the second lubricant are inert to ultraviolet light and do not interfere with ultraviolet light;
or, the first lubricant is selected from one or more of white vaseline, silicone oil, paraffin, mineral oil and grease.

4. The method of claim 1, wherein the film-forming liquid comprises one or more of an imidazole ionic liquid, a pyridine ionic liquid, a quaternary ammonium salt ionic liquid, a quaternary phosphine ionic liquid and a pyrrolidine ionic liquid;
or, the film-forming liquid comprises one or more of 1-ethyl-3-methylimidazolium tetrafluoroborate, 1-ethyl-3-methylimidazolium chloride, N-ethyl pyridine bromide, tributylmethylammonium chloride, tributylethylphosphonium bromide and N-butyl-N-methyl pyrrolidine bromide.

5. The method of claim 4, wherein the cover plate is a glass plate or a hard plastic plate that is resistant to ultraviolet light;
or, the second lubricant is selected from white vaseline, silicone oil, paraffin, mineral oil, and grease.

6. The method of claim 1, wherein the polymerization reaction is carried out under the irradiation of ultraviolet light;
the wavelength of the ultraviolet light is 250 nm-400 nm; and
the irradiation time of the ultraviolet light is 15 min-30 min.

7. The method of claim 1, wherein the polymerization reaction is carried out at a temperature of 20° C. -60° C.;
or, the polymerization reaction is initiated by an initiator selected from the group consisting of photoinitiator 907, photoinitiator 184, azobisisobutyronitrile, 2,4,6-(trimethylbenzoyl) diphenylphosphine oxide or benzoin and derivatives thereof.

8. The method of claim 1, wherein separating the polyion thin film comprises: placing the cover plate attached with the polyion thin film into water for standing;
or, the cleaning comprises ultrasonic cleaning in clear water, absolute ethyl alcohol and clear water in turn.

9. The method of claim 1, further comprising: wetting the substrate with a solvent before laying the tinfoil on the substrate;
the solvent is water, ethyl alcohol or a mixed solvent thereof.

10. The method of claim 1, wherein the film-forming liquid is prepared by the following steps:
step a, fully mixing the ionic liquid monomers; and
step b, adding a cross-linking agent and an initiator, and then carrying out an ultrasonic treatment to obtain the film-forming liquid.

11. The method of claim 10, wherein the step a further comprises: performing ultrasonic treatment on obtained mixed solution after the ionic liquid monomers are fully mixed; and the ultrasonic treatment is performed for 10 min-30 min; and
wherein the step b, the ultrasonic treatment is performed for 10 min-30 min.

12. The method of claim 10, wherein the ionic liquid monomers comprise bromobutane, vinylimidazole and acrylonitrile;
or, a molar ratio of the bromobutane to the vinylimidazole is 2:1 to 1:1;
or, the mass of the acrylonitrile is greater than or equal to the sum of the mass of the bromobutane and the vinylimidazol.

13. The method of claim 12, wherein the cross-linking agent is N, N-Methylenebisacrylamide, and the initiator is 2,4,6-(trimethylbenzoyl) diphenylphosphine oxide; or, the mass of the cross-linking agent is 8wt%-12wt% of the total mass of the bromobutane, the vinylimidazole and the acrylonitrile, and the mass of the initiator is 0.5 wt%-2 wt% of the total mass of the bromobutane, the vinylimidazole and the acrylonitrile.

14. The method of claim 10, wherein the film-forming liquid is prepared by the following steps:
mixing the bromobutane and the vinylimidazole in equal molar ratio, and carrying out an ultrasonic treatment on obtained mixed solution for 15 min until the two are fully mixed; adding an amount of the acrylonitrile equal to the total mass of the bromobutane and the vinylimidazole; adding an amount of N, N-Methylenebisacrylamide that is 8 wt% of the total mass of the
bromobutane, the vinylimidazole and the acrylonitrile and an amount of 2,4,6-(trimethylbenzoyl) diphenylphosphine oxide that is 1.0 wt% of the total mass of the bromobutane, the vinylimidazole and the acrylonitrile, and then carrying out the ultrasonic treatment for 15 min to obtain the film-forming liquid.

15. The method of claim 1, further comprising:
immersing the polyion thin film in a dye solution with a dye;
the dye is selected from one of bromocresol green, cresol red and methyl orange;
or, the dye solution is a solution obtained by dissolving the dye in a mixed solvent of water and ethyl alcohol, wherein in the mixed solvent of water and ethyl alcohol, the volume ratio of water to ethyl alcohol is 4:1-1:8, or 1:4;
or, in the dye solution, the concentration of the dye is 0.5-8 mg/ml, or 3.95 mg/ml.

16. A polyion thin film, wherein the polyion thin film is prepared by the method of claim 15.

17. A detection kit, comprising the polyion thin film of claim 16.

18. The kit of claim 17, wherein the detection kit further comprises a colorimetric card.
